(19) United States
(12) Patent Application Publication
(10) Pub. No.: US 2003/0096277 A1
(43) Pub. Date: May 22, 2003

(54) Allele specific PCR for genotyping

(76) Inventor: Xiangning Chen, Richmond, VA (US)

Correspondence Address: Whitham, Curtis & Christofferson, P.C., Suite 340, 11491 Sunset Hills Road, Reston, VA, 20190, US

(21) Appl. No.: 10/231,381

(22) Filed: Aug. 30, 2002

Related U.S. Application Data
(60) Provisional application No. 60/315,776, filed on Aug. 30, 2001.

(51) Int. Cl. C12Q 1/68; C12P 19/34

(52) U.S. Cl. 435/6; 435/91.2

(57) ABSTRACT

Methodology for a two-level allele-specific extension (AS-PCR) reaction for use in ultra-high throughput genotyping is provided. A primary AS-PCR reaction is carried out with primers containing both sequence-specific and artificial, universal domains. The primers are designed to render the PCR products 1) allele specific and 2) amenable to amplification with secondary primers which also contain universal domains. The secondary primers are designed to maintain allele-specificity, and to provide a detectable label.

### BACKGROUND OF THE INVENTION

1. Field of the Invention

The invention generally relates to high-throughput genotyping technology. In particular, the invention provides a method which utilizes two allele specific PCR (AS-PCR) reactions to amplify and identify a locus.

2. Background of the Invention

A great number of single nucleotide polymorphisms (SNPs) have been made publicly available by the Human Genome project and the SNP Consortium (1). To take full advantage of the SNP resources for genotyping, it is necessary to have available cost-effective and versatile methodology. Despite rapid progress in the field in recent years (4), a technology that is both cost effective and that does not require dedicated instrumentation is not yet available (2, 3). Popular methods such as single base extension with mass spectrometry detection (5, 6), pyrosequencing (7), the 5′ nuclease assay (8, 9) and the Invader assay (10, 11) all suffer from the limitation that they require specialized instrumentation. Commercially available products like SNaPshot (Applied BioSystems, Foster City, Calif.) and SNuPe (Amersham Pharmacia BioSciences, Piscataway, N.J.) make use of popular DNA sequencers but have limited multiplexing capacity due to length limit of extension primers. In order to make use of DNA sequencers more efficiently it will be necessary to find new ways to obtain longer primers for the extension reaction or to devise an efficient way to obtain allele specific products with a wide range of sizes.

The biochemistry of allele discrimination includes three categories: discrimination based on the properties of DNA polymerases, that are based on properties of DNA ligases and DNA hybridization (2, 12). Of them, methods based on the properties of DNA polymerases are the most popular. Several properties of DNA polymerases have been exploited for SNP genotyping, primer extension being a popular example. Technically, primer extension can be performed in two ways: one is to anneal an extension primer immediately upstream to the target polymorphism; the other is to design allele specific extension primers with the 3′ base matching the polymorphic target. The former approach identifies polymorphism by identifying the bases extended. Since the identification of the target polymorphisms needs only one base extension, this approach is known as single base extension (SBE) or minisequencing. The latter approach infers the polymorphism by detecting the products of extension from the allele specific primers. Allele specific PCR (AS-PCR) is based on this principle. It is a useful technique that has been exploited for SNP genotyping by several groups (13-15). Compared to popular SBE, AS-PCR has certain advantages. For example, it is a single step reaction, DNA amplification and allele discrimination are combined together, and its products are suitable for analysis by DNA sequencers. AS-PCR also has certain limitations, e.g. some SNPs may not be amenable to AS-PCR and their allele discrimination might not be optimal. Part of the problem originates from the 3′ mismatch bases of the allele specific primers. For some SNP markers, the mismatch of 3′ allele specific bases may not be sufficient to block the extension of DNA polymerases, making it difficult to distinguish the two alleles. However, when AS-PCR is performed and assayed kinetically there is a clear difference between the matched and mismatched primers and alleles can be identified reliably (16, 17). The different outcomes from end-point and kinetic assays suggests that multiple cycles of thermal amplification tend to blur the distinctions. It is therefore possible that by limiting the number of cycles, the clear difference between matched and mismatched primers may be reserved. Furthermore, recent reports of the use of the locked nucleic acid (LNA) (18-20) in oligonucleotides suggest that LNA may improve the performance of allele specific PCR.

It would be highly desirable to have available methods for high-throughput genotyping that are cost effective, highly discriminating, and readily amenable to analysis using common laboratory equipment.

### SUMMARY OF THE INVENTION

The present invention provides a new allele specific PCR (AS-PCR) design that utilizes widely available DNA sequencers for SNP genotyping. The design couples two AS-PCR reactions, and is therefore named AS-PCR², and produces labeled, allele specific products. In the AS-PCR² design, the primary AS-PCR is dedicated for allele discrimination with limited amplification and the secondary AS-PCR, which is artificially introduced, for product amplification. The separation of allele discrimination and product amplification overcomes the weakness of allele discrimination of regular AS-PCR and makes AS-PCR² a viable choice for general use for SNP genotyping.

In one embodiment, the invention provides a method of genotyping one or more loci in a DNA sample. The method includes the steps of

1) combining a sample containing

i) single stranded DNA or double stranded DNA, ii) at least one primary primer specific for one locus on one strand of DNA in the sample (the primary primer has a first homologous portion which hybridizes to one strand of DNA and a non-homologous portion which does not hybridize to the one strand of DNA) and at least one secondary primer having a second homologous portion which includes the sequences of the non-homologous portion of said primary primer;

2) conducting polymerase chain reaction (PCR); and

3) identifying amplicons of the PCR which include the non-homologous portion. The step of identifying allows the genotype of the one or more loci to be established.

In one embodiment of the method, the combining step is performed using a plurality of primary primers, each of which is specific for a different locus in the DNA sample. Each of the primary primers includes a homologous portion which is different for each primary primer, and an non-homologous portion which is identical for each primary primer. As a result of the presence of the individual homologous portions, each primary primer hybridizes to the DNA at a different locus. The non-homologous portion of the primary primers provides a mechanism for amplification of multiple loci and reduces cost to label allele specific products.

The non-homologous portion in the PCR products (amplicons) may be identified by any of several techniques including but not limited to electrophoresis, microarray detection, fluorescence polarization, fluorescence resonance energy transfer, and mass spectrometry.

The loci which are geneotyped may contain a variety of detectable distinguishing features which include but are not limited to SNPs, deletions, insertions, and short tandem repeats.

The secondary primer may contain a detectable label such as fluorescent dyes, antibodies, enzymes, magnetic moieties, electronic markers, and mass tags.

The invention further provides a primer set for genotyping one or more loci in a DNA sample. The primer set includes at least one primary primer specific for one locus on one strand of DNA in the sample, in which the primary primer has a first homologous portion which hybridizes to the one strand of DNA and a non-homologous portion which does not hybridize to the one strand of DNA, and at least one secondary primer having a second homologous portion which includes the sequences of the non-homologous portion of the primary primer. The secondary primer may contain a detectable label such as fluorescent dyes, antibodies, enzymes, magnetic moieties, electronic markers, and mass tags.

### BRIEF DESCRIPTION OF THE DRAWINGS

FIG. 1. Schematic representation of primary 10 and secondary 20 primers for use in the present invention. 11 represents the specificity domain and 12 represents the artificial domain of primary primer 10.

FIG. 2. Detailed schematic representation of AS-PCR² showing primary primers 10A, 10B and 30, and secondary primers 20A, 20B, and 40. Two consecutive AS-PCRs are coupled with coupling elements to increase allele discrimination and amplification efficiency. Limited amplification from the primary reactions produces enough templates for the secondary reactions and reduces errors from the primary reactions. The function of the secondary reactions is to generate sufficient products for detection by DNA sequencers or other detection systems. The separation of the functions of allele discrimination and product amplification makes it possible to achieve high specificity and amplification efficiency for AS-PCR², therefore, makes AS-PCR² viable for general use for SNP genotyping.

FIG. 3. Allele specific coupling of the primary and secondary AS-PCRs. Genomic DNA samples with known genotypes for marker SC_{—}31 were first amplified with only one allele specific primary primer that complemented the genotypes. The secondary primers for both alleles were then used to test the specificity of the secondary reaction. The dotted line peaks in the panels are GeneScan 500 ROX size ladders. The marker SC_{—}31 generates products of 196 bases. In panel A and B primer SC_{—}31 and SC_{—}33 were used to amplify homozygous G/G samples for the primary reaction. For panel C and D primer SC_{—}32 and SC_{—}33 were used to amplify homozygous C/C samples. The data presented illustrate that coupling elements consisting of 2 bases linked the primary and secondary reactions allele-specifically without detectable mismatch extension.

FIG. 4. The use of secondary primers of different length could simplify genotype scoring for heterozygous samples. Secondary primers of different length, 20 (SC_{—}40) and 23 (SC_{—}5) bases, labeled with R6G and BTMR respectively, were used to perform AS-PCR² for a SNP marker. The expected product size for the R6G labeled primer was 256 bp (gray line), that for the BTMR labeled was 259 bp (black line). As shown in the figure, the upper panel was a heterozygous sample, where two peaks 3 bases apart were clearly seen. The bottom panel was a homozygous sample, only one black peak was seen. The additional, smaller peaks in both panels were GeneScan 500 ROX size markers. The results illustrated clearly that the use of secondary primers of different length had simplified the genotype scoring for the heterozygous sample.

FIG. 5. The impact of the ratio of reporting dyes on the scoring of genotypes. AS-PCR² were performed for homozygous allele 1 (A/A, column A), heterozygous (A/G, column B) and homozygous allele 2 (G/G, column C) samples with varying ratios of the two reporting dyes (listed on the left) for the marker SC_{—}22. The products were analyzed by ABI 377 DNA sequencer using GeneScan software. The peak height ratio (BFL/BTMR) of the products (256 bases) was calculated from raw data for each sample and listed in the panel. When the ratio of the reporting dyes changed the peak height ratio also changes regardless of the genotypes. At optimal condition (row 3) the genotypes can be easily identified. But visual scoring of the peaks could be misleading when the ratio of reporting dyes was not optimal (row 1 or 5). Under these conditions it is essential to use systematic and sophisticated algorithms for genotype scoring.

FIG. 6. The impact of the ratio of reporting dyes on the ratio of peak height of the two alleles. The ratios of the two reporting dyes and the ratios of peak height from FIG. 5 were plotted. It was clear that when the ratios of the reporting dyes changed the ratios of peak heights also changed. Although the rate of change varied for each genotype group but the rate of change was constant within each group, as indicated by the correlation factor (R²) listed in the figure. This implies that genotypes can be scored reliably even the ratio of the reporting dyes is suboptimal.

FIG. 7. Genotype scoring for the marker SC_{—}31. After Genescan ran raw data (peak name, size, peak height and scan number) were exported from the software. The log value of peak height ratio was plotted. For those samples that had only one color for the expected size, an arbitrary peak height ratio was used (10 for allele 1 and 0.1 for allele 2). The plot showed three distinct groups, corresponding to homozygous allele 1, heterozygous and homozygous allele 2 respectively.

FIG. 8. An example of genotype scoring based on cluster analysis. In the example 48 samples were genotyped by AS-PCR² and products were separated by an ABI 377 sequencer. The genotypes were assigned based on the Euclidian distances to the centroids of each group (solid green) and assuming the two colors were independent. In the plot, there were two samples scored as failures (solid diamonds) and one sample unscored (asterisk) due to low confidence. Allele 1 (pink squires) were mostly on the X axis, and allele 2 (red dots) were on the Y axis. The heterozygous (blue triangles) were along the diagonal. When a covariant model was assumed, the unscored sample and one of the failed samples (the one off the Y axis) were scored as heterozygous. The other one of the failures scored as allele 2. In this example, all samples were scored correctly as verified by another method. The models used have slightly different outcomes, reflecting the stringency and efficiency of the classification.

FIGS. 9A and 9B. LNA primers improved allele discrimination for AS-PCR². AS-PCR² primers were designed for marker SC_{—}25 with both regular oligos and LNA oligos, and experiments were performed with 48 DNA samples. The reactions were run on ABI 377 DNA sequencers and peak heights for both BFL and BTMR were exported and plotted. Panel A was the results from regular primers where genotypes could not be scored. Panel B, in contrast, was results from the LNA primers where three distinct groups were observed. They represented three genotypes, namely homozygous allele 1, heterozygous and homozygous allele 2 as labeled 11, 12 and 22 respectively in the figure. Samples that failed the AS-PCR² were labeled “F”. The genotype scores from the LNA primers were confirmed correctly by the FP-TDI method.

FIG. 10. Multiplexing of AS-PCR². Examples shown were 5×multiplex with SNP markers SC_{—}22, SC_{—}25, SC_{—}28, SC_{—}31, SC_{—}34, with expected product sizes of 256, 216, 281, 196-331 base pairs respectively. The red peaks were GeneScan 500 ROX markers, and their sizes were listed on top of the figure. Three samples were shown (panel A-C). The numbers listed by the peaks were peak heights, F for BFL, T for BTMR. The numbers were provided to estimate relative amounts of products among the multiplexed markets.

FIG. 11. Multiplexing improves allele discrimination in AS-PCR2. Results for marker SC_{—}25 were shown. In single-plex reaction, SC_{—}25 could not score any genotypes (see FIG. 9A). When it was included in a 5×multiplex reaction significant improvement of allele discrimination was observed. For two genotype groups, homozygous allele 1 and heterozygous, its results correlated with that from the LNA primers, and scored correctly. The difference of peak height ratio between the heterozygous and the homozygous allele 2 was marginal, but the trend was clear. The results from LNA primers were included for comparison.

### DETAILED DESCRIPTION OF THE PREFERRED EMBODIMENTS OF THE INVENTION

The present invention provides a new method of AS-PCR (denominated “AS-PCR²”) for high throughput genotyping. The new method is highly affordable, and makes use of popular and readily accessible DNA sequencers for analysis.

The method of the present invention introduces an artificial secondary AS-PCR and couples it with the primary AS-PCR in an allele specific fashion. The coupling of primary AS-PCR with secondary AS-PCR serves two purposes: one is to separate the two conflicting processes (allele discrimination and product amplification) in regular AS-PCR. The separation of the two functions limits the impact of undesired mismatch extension of error-prone primary reactions on the overall amplification. The second purpose is to engineer a universal secondary primer set that maximizes amplification efficiency, minimizes mismatch extension and reduces the fixed cost per SNP. Since the investigator retains complete control of the secondary AS-PCR primer design and conditions, they can be tested and optimized to obtain maximal discrimination and optimal amplification.

The present invention thus utilizes a two-level approach to the PCR amplification of a locus, for example, a single nucleotide polymorphism (SNP) locus. The first level is a primary AS-PCR which utilizes “chimeric” primary primers that are partly allele-specific in nature (i.e. one portion of a primary primer contains sequences based on the targeted locus and allele of interest and is thus target/marker specific), and partly “artificial”, (i.e. another portion of the primer contains sequences that are not based on the targeted locus and allele of interest). The primary AS-PCR is basically a regular AS-PCR in which a specially devised “tail” is attached to the 5′ end of the allele-specific forward and target-specific reverse primers. The second level of AS-PCR utilizes fully “artificial” secondary primers which are not in and of themselves, allele or target specific. Rather, they are designed to be complementary to the artificial portion of the primary primers. The secondary AS-PCR level primers are coupled to the primary AS-PCR reaction via the non-specific artificial region (coupling elements). The design of the primers is such that this coupling also renders the secondary forward primer allele specific. Neither the forward or reverse secondary primers is, however, target/marker specific.

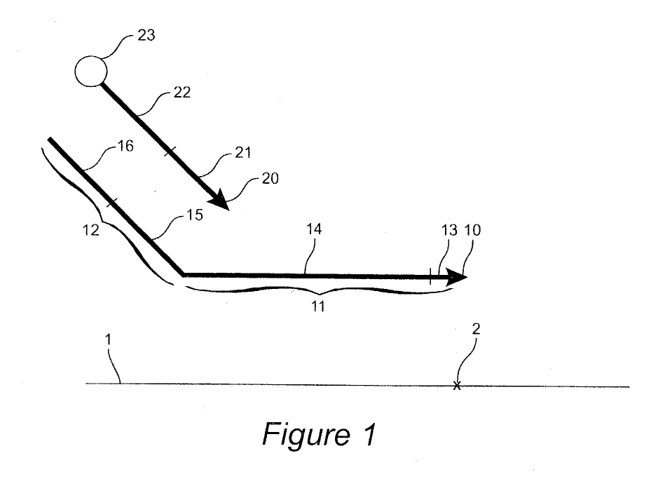

The fundamentals of this design for forward primers are illustrated in FIG. 1. FIG. 1 depicts a DNA strand 1 which contains a targeted locus possessing SNP site 2 (denoted by “X”). Primary primer 10 contains (3′) specificity domain 11 and (5′) artificial domain 12. (Note that for purposes of illustration a single generic primary primer 10 is depicted in FIG. 1; in the practice of the invention, several forward primary primers, one for each allele of the locus, and a reverse primer (or a plurality of reverse primers are used in a single reaction, as described in detail below.) Specificity domain 11 is on the 3′ end of the primer and contains sequences which are complementary to the sequence of the target site and one allele of interest. Specificity domain 11 itself contains two elements: allele element 13 (which may contain a single nucleotide representing an SNP variant and renders the forward primer specific for one allele) and target element 14. The sequence of target element 14 is complementary to sequences immediately 5′ to the SNP site and renders the primer specific for the targeted locus, but not necessarily for the allele. Allele specificity is conferred by allele element 13. In contrast, artificial element 12 (located on the 5′ end of primer 10) does not contain sequences based on the targeted locus. Instead, the sequence of artificial element 12 is tailored to facilitate secondary amplification, as is described in detail below. Artificial element 12 contains two elements: coupling element 15 and connecting element 16. Coupling element 15 contains sequences which “tag” the element as unique for a given allele. Coupling element 15 thus functions as a sort of “adapter” sequence between the first and second AS-PCR levels. As a result, the amplification product produced by a primary AS-PCR reaction of one allele will contain a 5′ “tail”sequence that is unique for that allele. Connecting element 16 is an artificial sequence designed to facilitate the second level of PCR amplification; it has sequences identical to those of the secondary forward primers. A PCR product from this primer will thus contain sequences complementary to secondary primer sequences.

A secondary primer 20 for use in the secondary PCR amplification reaction is also depicted in FIG. 1. Secondary primer 20 will amplify the PCR products of the primary AS-PCR reaction. Secondary primer 20 contains a single domain with three elements: a coupling element 21, the sequence of which is identical to the sequence of coupling element 15 of primary primer 10; a connecting element 22, the sequence of which is identical to the sequence of connecting element 16 of primary primer 10; and a detection element 23. The homology between coupling elements 15 and 21 renders secondary primer 20 allele specific.

Due to the homology between artificial domain 12 of primary primer 10 and secondary primer 20, the PCR products produced by amplification with primary primer 10 (i.e. the products of the primary AS-PCR amplification reaction) will be susceptible to amplification by secondary forward primer 20. Detection element 23 is a labeling or tagging moiety which serves to allow detection of PCR products which are amplified by the secondary AS-PCR reaction.

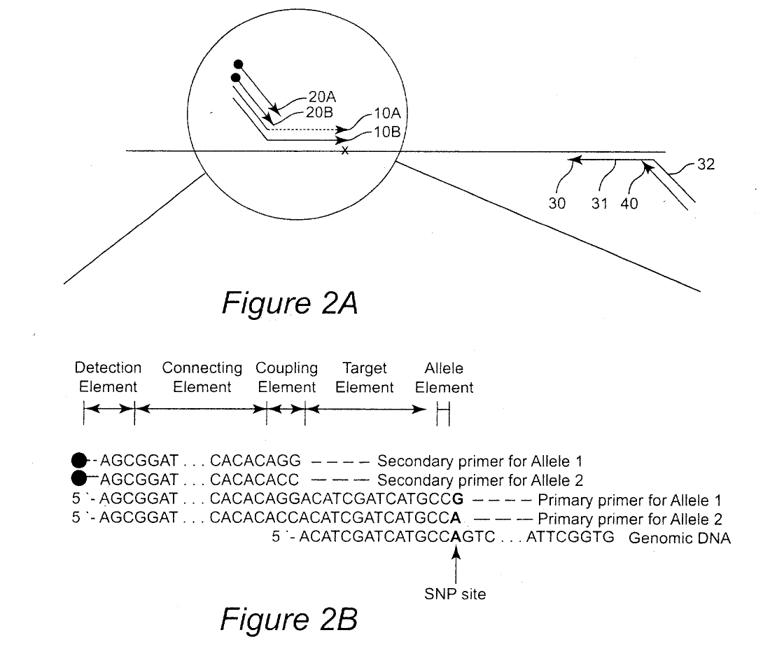

A detailed, expanded view of the primers of the present invention is depicted in FIG. 2. FIG. 2B depicts genomic DNA of an SNP locus of interest which has two known alleles. Allele 1 (not shown) has the nucleotide G at the SNP site and Allele 2 (shown) has the nucleotide A at the SNP site. As can be seen, for a single AS-PCR² reaction at this locus, both primary and secondary AS-PCR levels use three primers (FIG. 2B). The primary AS-PCR level uses two forward primers 10A and 10B (located 5′ to the SNP of interest, denoted by “X”) and one reverse primer 30, located 3′ to the SNP. For the secondary AS-PCR level, forward secondary primers 20A and 20B and secondary reverse primer 40 are used to amplify the PCR products of the primary AS-PCR reaction.

As described above, with reference to FIG. 1, the primary and secondary primer domains contain various elements. The elements can be understood in detail with reference to FIG. 2B, particularly with reference to the exemplary sequences.

I. Primary primers. Each primary primer 10A and 10B has two domains, (a specificity domain 11 and an artificial domain 12 as described for 10 of FIG. 1). The specificity domain functions to amplify the targeted sequences from genomic DNA in allele-specific fashion, and the artificial domain connects the primary AS-PCR to the secondary AS-PCR.

A. Specificity domain of primary primer. The specificity domain of a primary primer has two elements, i) the allele element (or allele-specific element) and ii) the target element or target-specific element (FIG. 2B).

i) The allele-specific element is on the 3′ end of the specificity domain. For SNPs, the allele-specific element is the one base that is complementary to the SNP bases at the targets. For insertion/deletion, this allele specific element is designed to amplify only one allele of the possible alleles. For microsatellite markers, the allele-specific element can be omitted because the alleles will be represented (i.e. distinguished from one another) by their length. The purpose of the allele element in the specificity domain is to specifically amplify only one allele (e.g. allele 1 in FIG. 2B) with one primer, and a second allele (e.g. allele 2 in FIG. 2B) with another primer.

ii) The target element is on the 5′ end of the specificity domain. The purpose of the target element in the specificity domain is to specifically anneal to the targeted genomic fragment. The design of the target element can follow the teaching of regular PCR primer design as is well-known to those of skill in the art.

B. Artificial domain of primary primer. The artificial domain of a primary primer is located at the 5′ end of the specificity domain and contains two elements: i) an allele-“coupling” element or coupling element, and ii) a connecting element.

i) The allele coupling element is deliberately designed to render the artificial domain of a given primary primer specific for an allele. (The specificity domain is already unique for an allele due to the sequence at the SNP site.) The artificial domain is rendered allele specific via the inclusion of a short sequence that is unique to the primer for a particular allele and which thus distinguishes one connecting domain from another in an allele specific fashion.

For example, in FIG. 2, the artificial domain of the primary primer for allele 1 is distinguished from the connecting domain of the primary primer for allele 2 by utilizing the dinucleotide sequence GG for the former and CC for the latter. This element therefore links the two AS-PCR reactions in an allele-specific fashion. The length of the coupling element can be, for example, about 1-10 bases or more depending on the particular design of multiplexing and the number of alleles involved (more alleles will require the ability to design more complex distinguishing sequences). The coupling element is thus allele specific, but not target specific. In an ideal situation, it should be designed to have the same Tₘ for differentiating alleles because if differing Tₘs are used for different alleles, the extension efficiencies of the secondary primers may be affected, resulting in different amounts of end products of the two alleles. Since genotypes are scored based on relative amounts of products from the two alleles, any factors that introduce variations would be less desired. The keys for the design of coupling elements are (1) to ensure allele-specific linkage between the primary and secondary reactions; (2) to eliminate mismatch extension; and (3) to maintain the balance of extension efficiencies among the alleles.

ii) The connecting element is at the 5′ end of the coupling element. The function of the connecting element is two-fold. It provides a template for the secondary primers and facilitates multiplexing reactions. In an ideal situation, the connecting element should have a Tₘ that is lower than that of the target element in the primary primers. The difference in Tₘs between the target element and the connecting element allows the primary and secondary reactions to be performed in separate temperature zones so that the two reactions do not interfere each other. For multiplex considerations the connecting sequences should be unique, should not form primer dimers, and should not self-prime.

II Secondary primers. The secondary primers are reusable and are designed to be common to all targets. Each secondary primer contains only a single artificial domain which contains three elements: i) the coupling element, ii) the connecting element and iii) the detection element. They have following features:

i) For a given allele, the coupling element has exactly the same sequence as the coupling element in the corresponding, allele-specific primary primer. This feature assures allele-specific connection between the primary and the secondary AS-PCR. When the primary reaction amplifies the genomic DNA, the amplified products will contain the sequence complementary to the coupling element, and it will serve as the template for the secondary reaction. The coupling element is allele-specific but not target-specific.

ii) The connecting element is located at the 5′ end of the coupling element. The connecting element has exactly the same sequence as the connecting element in the primary primers.

iii) The detection element is located at the 5′ end of the connecting element. The detection element is adjacent to the connecting element, which in turn is adjacent to the allele-specific coupling element of the secondary primer, and the coupling elements are linked to the allele elements which amplify allele-specific genomic DNA. Due to this linkage, the detection elements are, in effect, also allele specific, and identification of the detection elements permits identification of the alleles at the target sites of DNA samples.

The AS-PCR² methodology of the present invention may be utilized to effectively genotype a wide variety of polymorphisms, such as SNPs, short insertions and deletions, and microsatellite markers. Depending on the genetic marker under study and the detection mechanisms, the design of the primers may be modified in the following ways:

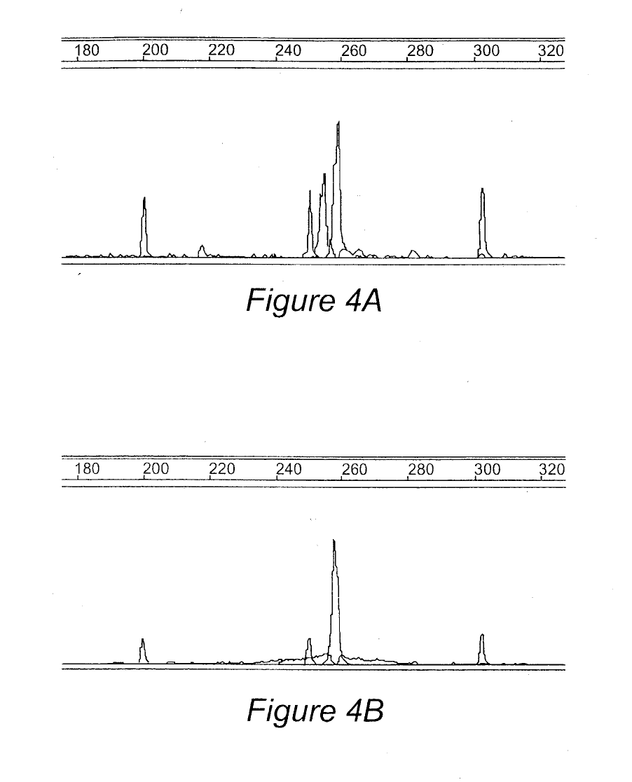

1). To genotype SNPs: When only one marker is tested, there would not be many restrains on the design of the target domain, so this could follow the teaching of PCR primer design. When multiple SNPs are tested together (multiplexing), then the sizes of the amplicons should be at least 3-5 bases apart for electrophoresis detection. The size restraint would not apply when another detection format is intended, such as microarray and mass spectrometry. As for the secondary primers there is an option for better allele scoring at the expense of multiplexing capacity when electrophoresis and mass spectrometry are used. The option is to design the two allele specific primers with different length. The offset of 1-3 bases would be sufficient (see Example 3 and FIG. 4).

2). To genotype microsatellites and short insertion and deletions: There is no need of the allele elements in the primary primers because the polymorphisms are represented as differences in length/size. The coupling elements would serve as tags for different markers.

Since the design of the secondary AS-PCR is artificial (i.e. the sequence of the primers used is not constrained by the sequence of the locus to be amplified, rather they can be adjusted as necessary or desirable), it is possible to test and optimize primers used in the secondary AS-PCR to obtain maximal discrimination and optimal amplification. The coupling of the primary AS-PCR with a secondary AS-PCR serves two purposes: one is to limit the undesired mismatch extension of the primary AS-PCR, and the other is to reduce the cost of genotyping. Those of skill in the art will recognize that many well-known methods exist and are routinely used in the design of primers that may be utilized in the practice of the present invention. Such primer design takes into account factors such as areas of homology, the desired Tm of the sequences which are to be hybridized, the number of complementary base pairs needed to effect hybridization of sufficient strength, length of sequences, potential for primer-dimer formation, potential for formation of secondary structure, intrastrand basepairing of ssDNA, and the like. Examples of programs intended to aid in the design of primers include, for example, Primer 3, Oligo, Primer Star and Primer Express, etc.

Further, “locked” nucleic acids (LNAs) may be used in the practice of the present invention. LNAs use a new nucleotide analog that uses a methylene linker to connect the 2′-O position to the 4′-C position of the ribose ring in a regular nucleotide. The LNA oligomers follow the Watson-Crick base pairing roles and hybridize to complementary oligonucleotides.

Oligomers that used LNA improved the performance of hybridization by forming more stable duplex structures (26, 27, 31, 32). With respect to the location of the primary reverse primers, in a preferred embodiment they are located about 100 to about 1000 base pairs downstream from the forward primary primers, giving PCR products in the size range of about 150 to about 1000 bps.

Those of skill in the art will recognize that, in order to perform higher levels of multiplexing using the method of the present invention, the reverse primer in the primary reaction may also have a sequence tag similar to that taught by Shuber (U.S. Pat. No. 5,882,856, the complete contents of which are hereby incorporated by reference) but with one important difference. In the practice of the present invention, the Tm does not have to be higher than that of the target domain. When the primary and secondary reactions are performed together it is actually preferred to have a lower Tm for the artificial tag, for this allow the performance of two reactions at separate temperature zones. Although both ours and Shuber's designs are intended to facilitate multiplexing PCR, the two approaches accomplish the goal by different mechanisms. Shuber's design relies on the higher Tm of the second domain to function as annealing nuclei and the nuclei then extend rapidly to the first domain. This zipping function of the nuclei helps to narrow the annealing temperature of different primers so that they could achieve more even amplification. The design of the present invention takes a different approach. In the present invention, the primary reaction is to make a limited but even amount of templates for the secondary reaction. This is accomplished by using equal but a limited amount of primary primers. In a closed system when the more robust primers are used up, the DNA polymerases are forced to work with the less robust primers. In the end, even the less robust primers would produce equal amount of templates for the secondary reaction. The secondary reaction of the present invention is a reaction that uses only one set of primers to amplify all targets in the multiplex. Because there is only one primer set, the primers function as in a simple PCR, all amplicons are amplified equally. The reverse primer for the secondary reaction has exactly the same sequence as the sequence tag in the reverse primer of the primary reaction.

Those of skill in the art will recognize that the amount of PCR products obtained from different loci during the primary amplification are not necessarily equal. However, the technique promotes amplification of loci that might not otherwise be amplified at all, or might be amplified at a very low level. PCR products from these otherwise difficult to amplify loci are thus obtained at readily detectable levels. See, for example, Example 7.

Further, one or more restriction enzyme recognition sites may be incorporated into the primers as necessary, e.g. into the sequence tag of the reverse primer for usage in conjunction with electrophoretic detection. The use of a restriction enzyme prior to electrophoresis would make the size of the products of the secondary reaction very precise, thereby increasing the resolution and the capacity for multiplexing.

The detection elements which are incorporated into the secondary forward primers can be any detectible moieties that provide a mechanism for their detection, including but not limited to fluorescence, antibody, enzyme, magnetic, electronic, mass tag, or detectable moieties of other natures.

The primary and secondary reactions of the present invention can be performed separately or combined together. When the two reactions are combined, the Tₘ for the primary and the secondary primers should be designed to be different. The Tₘ difference between the primary and secondary primers provides an opportunity to perform the two reactions at different temperature zones. For example, one can use a higher annealing temperature to amplify target DNA using the primary primers. When sufficient amount of products from the primary reaction are accumulated, one then lowers the annealing temperature for the secondary reaction. For example, one could use 70° C. as the annealing temperature for the primary reaction and cycle 10 times, then the temperature could be lowered to 50° C. for 30 more cycles.

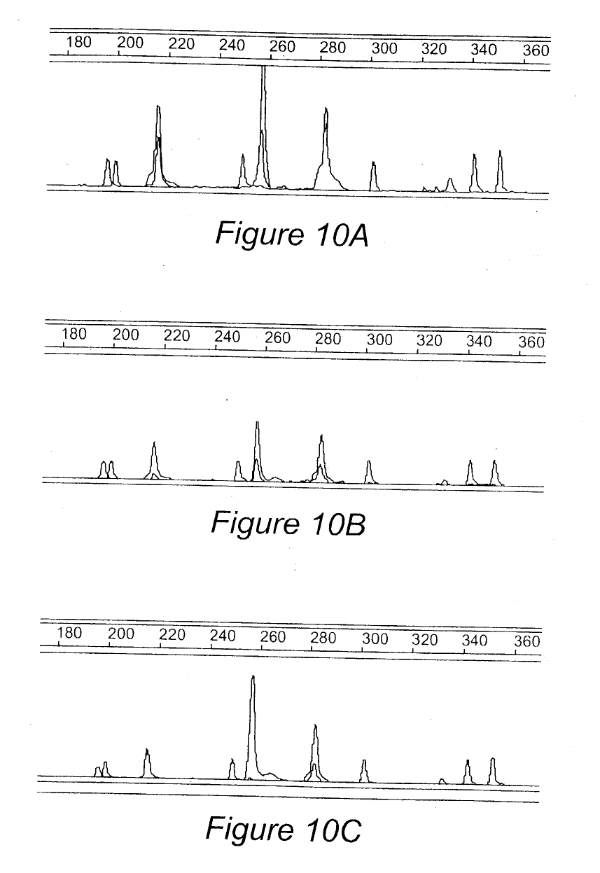

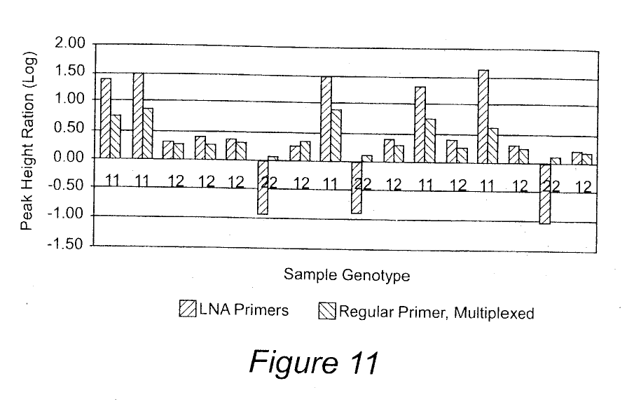

AS-PCR is dynamic, the two allele specific primers compete against each other. In a closed system more competition tends to amplify the difference among the competitors. For that reason, multiplexing AS-PCR would intensify the competition and make the differences between the two alleles more dramatic. In other words, multiplexing AS-PCR would improve the allele discrimination. This principle is illustrated by the data presented in Example 6 (see FIGS. 10 and 11).

There is another way to improve allele specificity, that is to reduce the number of cycles in the primary reaction. In the AS-PCR design of the present invention, all mismatch extension originates from the primary reaction. Therefore, when the number of cycles is reduced in the primary reaction, there is less opportunity for mismatch to occur. Our stepwise AS-PCR² design resolves the two conflicting processes that occur in regular AS-PCR, namely, allele discrimination and product amplification. This can be accomplished by using a low but equal concentration of primary primers along with limited cycling. For the primary reaction, the discrimination derives from one base mismatch at the SNP site; therefore, the discriminating power is limited. In the secondary reaction, primers with 2-3 or more mismatched bases can be designed, therefore, increasing the discriminating power between the alleles. For example, one can use only about 0.1 to about 0.5 nM of primary primers (roughly about 1% to about 5% of the amount for regular PCR) to amplify the target genomic fragment by about 5 to about 10 cycles. Then, one would use a non-limiting amount (e.g. about 25 to about 50 nM) of secondary primers to amplify the products from the primary reaction. Combining these two levels of discrimination means that AS-PCR² is much more specific than conventional allele specific PCR.

The products from the secondary reaction are the products to be detected. Depending on the nature of the detection tag, the detection methods can vary considerably. Following is a partial list of methods that can be used for the detection of the secondary products:

Electrophoresis: This category covers broad range, including but not limited to slab gel, sequencing gel, capillary electrophoresis, microfluidics, microarray electrophoresis etc. This group is of particular interest for high throughput and accessibility, because it allows a high level of multiplexing in both PCR and detection, and there are a variety of instruments for electrophoresis available in academic and industrial laboratories. Electrophoretic separation depends on the sizes and the labeling of the AS-PCR² products. As long as the sizes and colors of the products are not exactly the same, electrophoresis would be able to separate them. For example, the BODIPY series of fluorescent dyes have been shown to minimize emission overlaps between dyes (Metzker 1996). Examples of other dyes which may be utilized in the practice of the present invention include but are not limited to FAM, fluorescein, R110, R6G, TAMRA, ROX, Texas red, Cy3 and Cy5,etc.

Microarray detection: The AS-PCR² technique produces fluorescence labeled PCR products when the detection tags are fluorescence groups. When these products are hybridized to complementary oligonucleotides on a microarray, the separation of each amplicon will be achieved. Detecting the colors and fluorescence intensities at a given array address that has oligonucleotides complementary to a specific marker will enable scoring the genotypes of a DNA sample. Because microarray separation does not depend on the sizes of AS-PCR² products, this will release some restraints on the design of AS-PCR² primers. Because of the availability of high density microarrays the capacity of throughput is very high, on the order of 10⁵ genotypes per day.

Fluorescence polarization (FP): When the detection tag is a fluorescence label, FP can be used as detection mechanism. For FP, the fluorescence labeled secondary primers are relatively small compared to the products of extension. Because of the change of molecular mass during the reaction, the FP property would also change. By detecting the change in FP property, the genotypes of specific alleles can be determined. To make this detection format more attractive, a special protein binding sequence can be used as the connecting element. The binding of a high volume protein to the connecting element when it becomes double stranded (e.g. amplified) would improve the separation. Examples of such proteins include but are not limited to T3, T7 DNA polymerases, and exonucleases VII.

Fluorescence resonance energy transfer (FRET): In this particular application, the fluorescence dye on the secondary primers acts as a receptor. A common donor such as a dye labeled dNTP, may be employed. When the donor is incorporated onto the dye-labeled secondary primers, FRET would occur. By detecting the occurrence of FRET, the genotypes of the samples can be inferred.

Mass spectrometry: Mass spectrometry measures molecular mass. In the practice of the present invention, when the secondary extensions occur, the mass of the secondary primers changes. By detecting the change, the genotypes of the samples may be determined.

The methods of the present invention can be utilized to amplify a single genetic locus of interest. However, the primary intent is multiplex amplification of several loci at once. For single locus detection, individual primary primers are designed for each locus. The secondary primers, being universal in nature, can be used for more than one locus. When multiple loci are amplified, the primers (both primary and secondary primers) are designed in such a way that the size of the secondary PCR products are distinguishable by size or by mass. Multiplex AS-PCR² is further discussed in Example 6 below.

To score genotypes of AS-PCR² products by DNA sequencers it is necessary to identify from which allele specific primer the products were generated. If a sample is heterozygous, one expects to see a band with two colors because products from both alleles have same size. Because a mismatched primer does extend and because matrix spectral correction is not complete, a homozygote can be seen to have two colors. This could complicate the genotype scoring. One simple solution to resolve this problem is to use two allele specific primers of different lengths. In this way the products from the two alleles would have different sizes, i.e. they would be offset. By doing this the scoring of a heterozygote is transformed from measuring the peak height ratio of a peak to counting the number of peaks. This procedure, therefore, would make the scoring simpler and more reliable. Example 3 further describes such a design strategy.

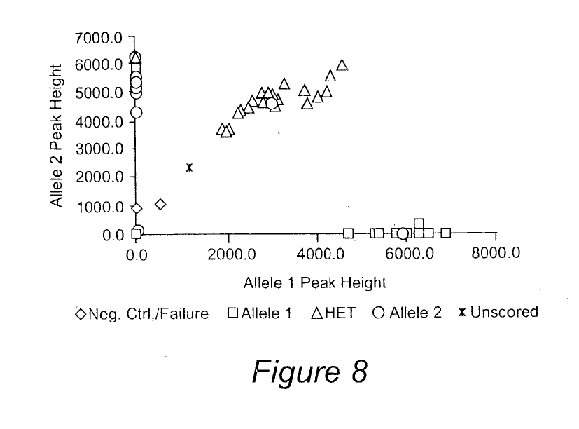

Those of skill in the art will recognize that there are several ways to analyze the data obtained from an AS-PCR² genotyping reaction. For example, one may take a ratio of intensities of the reporting dyes as indexed by the peak heights. Each genotype group would have a distinct ratio even if there was a small fraction of mismatch extension. Alternatively, it is possible to plot the intensities of the reporting dyes in a two dimensional plot, and to use distance-based cluster analysis to classify the groups. To begin, an independent model is assumed, and Euclidian distances are calculated between the samples and the initial centroids of each potential group. The coordinates of the initial centers can be estimated by the frequency distribution of the samples, or assigned arbitrarily. The samples are then assigned to a group based on their minimal distances. Then the coordinates of the centers for each group are recalculated based on the membership data points assigned from the first round classification. After several rounds of calculation, the true centers of each group can be established and used for final genotypic classification. FIG. 8 of Example 4 shows an example of this analysis. For more sophisticated analysis, other transformed distances and covariance models can be used. For each classified sample, the posterior probability of group membership (i.e., genotype) can be calculated to provide a confidence measure for the genotypes assigned.

Those of skill in the art will recognize that the methods of the present invention will have wide applicability for high throughput genotyping. Any locus or group of loci of interest may be so amplified by these methods. To facilitate such endeavors, the invention also provides a kit which includes a secondary PCR primer set and instructions for the design and use of primary primers which are compatible for use with the secondary primer set. For example, the secondary primers possess sequences which are the equivalent of the “second homologous portion” described above. The instructions would describe the sequence of the second homologous portion so that the user could design primary primers containing: 1) a first homologous portion that hybridizes to a sequence of interest (e.g. the flanking region of a locus of interest) and 2) a first non-homologous region identical in sequence to the second homologous portion of the secondary primers in the kit. The “generic” secondary primers (which are present in optimized amounts) can be used in a second round of PCR amplification to amplify the PCR products produced by the primary primers in a first round of amplification. Alternatively, first and second rounds of amplification may be carried out concomitantly.

### EXAMPLES

### Methods

Regular oligonucleotides used in this study were obtained from Life Technologies, Inc. (Grand Island, N.Y.). Fluorescence labeled primers, SC_{—}4 and SC_{—}5, were purified by HPLC. SC_{—}4 was labeled with BODIPY-fluorescein (BFL) at its 5′ end. SC_{—}5 was labeled with BODIPY-TAMRA (BTMR) at its 5′ end. The sequences of the primers used in this study are listed in Table 1. AS-PCRs were performed in MJ Research Tetrad DNA Engine in 12 μL of volume in two sequential reactions. The initial reaction mixture containing 10 mM of Tris-HCl, pH 8.3, 50 mM of KCl, 2.5 mM of MgCl₂, 0.25 mM of each dNTPs, 0.5 nM of each primary primers (SC_{—}22, SC_{—}23 and SC_{—}24 for marker SC_{—}22, and SC_{—}31, SC_{—}32, SC_{—}33 for marker SC_{—}31), 75 ng of genomic DNA and 0.5 U of AmpliTaq Gold DNA polymerases. The thermal cycling conditions were 95° C. for 10 min followed by 10 cycles of 95° C. for 30 sec, 65° C. for 5 sec, ramp at −0.1° C./sec to 55° C., 55° C. for 1.5 min. After the primary reaction, 25 nM of SC 4, 25 nM of SC_{—}5 and 50 nM of SC_{—}6 in a volume of 2 μL were added to each reaction. The secondary reaction used the conditions of 25 cycles of 95° C. for 30 sec, 60° C. for 1.5 min with a final extension at 72° C. for 10 min. LNA primers were synthesized by Proligo LLC (Boulder, Colo.).

TABLE 1
Primer sequences used in the experiments

| Oligo | | Product | | |
|---|---|---|---|---|
| ID | Sequence | Length | Modification | SEQ ID NO. |
| SC 4 | AGCGGATAACAATTTCACAC | | 5′ bodipy | SEQ ID NO. 1 |
| | AGG | | fluorescein | |
| SC 5 | AGCGGATAACAATTTCACAC | | 5′ bodipy | SEQ ID NO. 2 |
| | ACC | | TAMRA | |
| SC 6 | CCCAGTCACGACGTTGTAAA | | None | SEQ ID NO. 3 |
| | ACG | | | |
| SC 22 | CCCAGTCACGACGTTGTAAA | 256 | None | SEQ ID NO. 4 |
| | ACGcttacgcataaacccccaag | | | |
| SC 23 | AGCGGATAACAATTTCACAC | 256 | None | SEQ ID NO. 5 |
| | AGGagcagactcaaatggatttctggA | | | |
| SC 24 | AGCGGATAACAATTTCACAC | 256 | None | SEQ ID NO. 6 |
| | ACCagcagactcaaatggatttctggG | | | |
| SC 25 | AGCGGATAACAATTTCACAC | 216 | None | SEQ ID NO. 7 |
| | AGGtcctccagaggctgaggtG | | | |
| SC 26 | AGCGGATAACAATTTCACAC | 216 | None | SEQ ID NO. 8 |
| | ACCtcctccagaggctgaggtA | | | |
| SC 27 | CCCAGTCACGACGTTGTAAA | 216 | None | SEQ ID NO. 9 |
| | ACGagcatttcagactcccagt | | | |
| SC 28 | AGCGGATAACAATTTCACAC | 281 | None | SEQ ID NO. |
| | AGGgtacactaaggtgggagtaatT | | | 10 |
| SC 29 | AGCGGATAACAATTTCACAC | 281 | None | SEQ ID NO. |
| | ACCgtacactaaggtgggagtaatC | | | 11 |
| SC 30 | CCCAGTCACGACGTTGTAAA | 281 | None | SEQ ID NO. |
| | ACGatcacttcaccccacacac | | | 12 |
| SC 31 | CCCAGTCACGACGTTGTAAA | 196 | None | SEQ ID NO. |
| | ACGgcacgatactgaatgcacca | | | 13 |
| SC 32 | AGCGGATAACAATTTCACAC | 196 | None | SEQ ID NO. |
| | AGGgacatggtcttaaaatgtataaaaG | | | 14 |
| SC 33 | AGCGGATAACAATTTCACAC | 196 | None | SEQ ID NO. |
| | ACCgacatggtcttaaaatgtataaaaC | | | 15 |
| SC 34 | CCCAGTCACGACGTTGTAAA | 331 | None | SEQ ID NO. |
| | ACGatccatgagggttggaatca | | | 16 |
| SC 35 | AGCGGATAACAATTTCACAC | 331 | None | SEQ ID NO. |
| | AGGttaacattgttttcatcgcccactaaT | | | 17 |
| SC 36 | AGCGGATAACAATTTCACAC | 331 | None | SEQ ID NO. |
| | ACCttaacattgttttcatgcccactaaC | | | 18 |
| SC40 | GGATAACAATTTCACACAGG | 276 | 5′ bodipy | SEQ ID NO. |
| | | | R6G | 19 |
| SC172 | CGGATAACAATTTCACACAG | | | SEQ ID NO. |
| | GtcctccagaggctgaggtG | | | 20 |
| SC173 | CGGATAACAATTTCACACACC | | | SEQ ID NO. |
| | tcctccagaggctgaggtA | | | 21 |
| SC27 | CCCAGTCACGACGTTGTAAA | | | SEQ ID NO. |
| | ACGagcatttcagcactcccagt | | | 22 |
| SC202 | GGATAACAATTTCACACAGGc | | | SEQ ID NO. |
| | cccagcctcccaaagcA | | | 23 |
| SC203 | GGATAACAATTTCACACACCC | | | SEQ ID NO. |
| | cccagcctcccaaagcG | | | 24 |
| SC9 | CCCAGTCACGACGTTGTAAA | | | SEQ ID NO. |
| | ACGcagattcggggcagaaaata | | | 25 |
| SC204 | GGATAACAATTTCACACAGGc | | | SEQ ID NO. |
| | agacggtcacccacatcA | | | 26 |
| SC205 | GGATAACAATTTCACACACCC | | | SEQ ID NO. |
| | agacggtcacccacatcC | | | 27 |
| SC13 | CCCAGTCACGACGTTGTAAA | | | SEQ ID NO. |
| | ACGccaacaatgagcgaattactga | | | 28 |
| SC208 | GGATAACAATTTCACACAGGc | | | SEQ ID NO. |
| | ctttcccaactgagcacA | | | 29 |
| SC209 | GGATAACAATTTCACACACCc | | | SEQ ID NO. |
| | ctttcccaactgagcacG | | | 30 |
| SC92 | CCCAGTCACGACGTTGTAAA | | | SEQ ID NO. |
| | ACGttcctgaagggatgagttcc | | | 31 |
| SC210 | GGATAACAATTTCACACAGGg | | | SEQ ID NO. |
| | tgtgccatgtcctgttcA | | | 32 |
| SC211 | GGATAACAATTTCACACACCg | | | SEQ ID NO. |
| | tgtgccatgtcctgttcG | | | 33 |
| SC113 | CCCAGTCACGACGTTGTAAA | | | SEQ ID NO. |
| | ACGcacccaaggcactatctcct | | | 34 |

* LNA bases are highlighted in bold.

In the experiments of reporting dye ratio optimization, the amount of secondary primers used varied from reaction to reaction as described in the experiments. The base amount of the primers was 25 nM. For example in the experiment that used a BFL/BTMR ratio of 1:1, 25 nM of each of SC_{—}4 and SC_{—}5 was used. When the ratio changed to 2:1, 50 nM of SC_{—}4 and 25 nM of SC_{—}5 were used. The amount of SC_{—}6 was kept constant at 75 nM.

GeneScan Run and Analysis

One microliter of AS-PCR products was mixed with loading buffer and GeneScan 500 size markers (ROX) and loaded on 6% sequencing gel. Samples were run on ABI 377 sequencer for 3 hours using GeneScan software. When the runs finished gel lanes were tracked, extracted and analyzed by the GeneScan software. For each sample raw data such as peak name, size, peak height, peak area, time appeared (in minutes) and scan number were exported for each color for genotype scoring.

Genotype Scoring

The raw data exported by the GeneScan software were used to score genotypes of samples. To score the genotype of a sample, peaks within a 2 base range of the expected product size were considered. If there was only one peak, either a blue peak (BFL) or a yellow peak (BTMR), in the expected size range the sample was scored as homozygous. A blue peak represented homozygous allele 1, a yellow peak represented homozygous allele 2. When both the blue and yellow peaks were presented in the expected size range, the scan number was used as the criteria to identify if they were the two alleles of the AS-PCR. When the difference of the scan number between the two peaks was less than or equal to 3 data points, it was considered that they were products from the same AS-PCR. Otherwise they were considered as being from different AS-PCRs. For those samples in which both blue and yellow peaks were observed in the expected size range, the ratio of peak height of the blue and yellow peaks was used to score the sample. The ratio varies slightly from marker to marker but was consistent for the same marker.

Introduction to Examples.

To demonstrate the principle of the invention, several SNPs that had previously been genotyped for another unrelated schizophrenia project were selected, and AS-PCR² primers for the SNPs were designed as described in the Detailed Description of the Invention. The sequences of primers used in the study are listed in Table 1 and 2. The following experiments were then carried out:

(a) coupling the two levels of AS-PCRs (Example 1);

(b) optimizing the system for detection by ABI 377 DNA sequencer (Example 2);

(c) scoring genotypes by secondary primers of different lengths (Example 3);

(d) performing genotyping comparison with the FP-TDI method (23) (Example 4);

(e) scoring genotypes for AS-PCR²,(Example 5);

(f) performing AS-PCR² with LNA primers; (Example 6); and

(g) multiplexing AS-PCR² (Example 7);.

### Example 1

### Coupling the Two Levels of AS-PCRs

As described above, in the practice of the present invention, an artificial secondary AS-PCR is introduced and coupled with the primary AS-PCR in an allele specific fashion. The coupling of primary AS-PCR with a secondary AS-PCR serves two purposes: one is to limit the undesired mismatch extension of primary AS-PCR, the other is to reduce the cost of genotyping. Primers of the secondary AS-PCR can be tested and optimized to obtain maximal discrimination and optimal amplification.

In this study M13 reverse primer was used as the connecting element and two bases (GG and CC) as the coupling elements. The use of two bases for the coupling elements were based on previous reports (21, 22) that two consecutive mismatch bases were sufficient to block the extension of DNA polymerases. In the practice of the present invention, the coupling elements serve two goals: i) to connect the primary and secondary reaction allele-specifically, and ii) to increase the overall allele discrimination. It is thus important to demonstrate that no mismatched extension occurs at the secondary reaction.

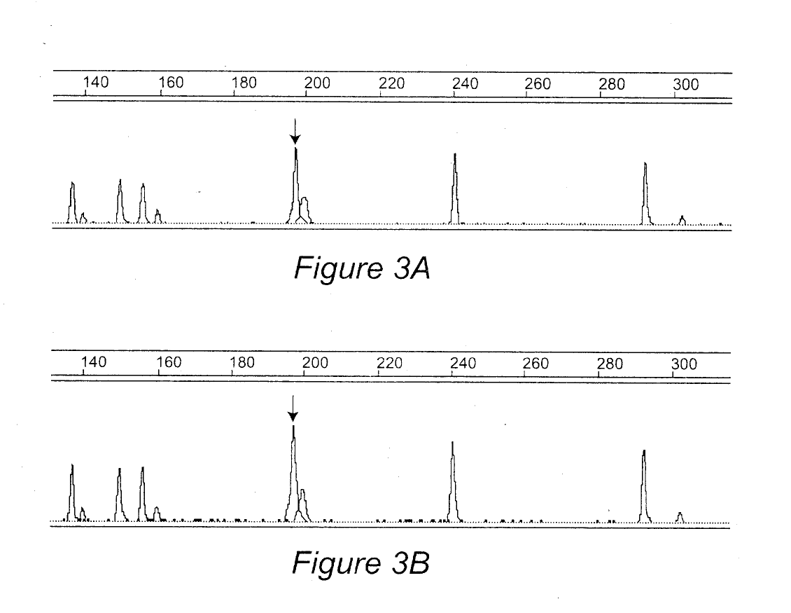

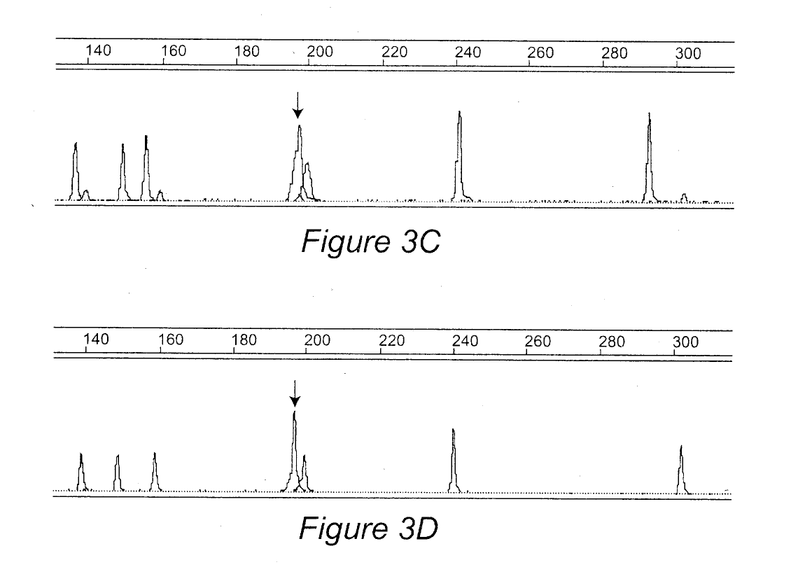

In order to do so, the following experiments were conducted: Several DNA samples with known genotypes for marker SC_{—}31, either homozygous allele 1 (G/G) or homozygous allele 2 (C/C), were chosen to for the experiments. In the primary reactions, only one of the two allele specific primers that matched the known genotypes of the DNA samples was used so that only one allele was amplified in the reactions. In the secondary reactions, both allele specific primers were used so the occurrence of mismatched extension could be detected. In these experiments, if the two base mismatches (the coupling element) between the primary and secondary primers were sufficient to block the extension of the mismatched primer, we would expect that only the matched primers would produce extension products. If products from both matched and mismatched primers were seen, the ratio of the two products would reflect the difference of efficiency between the matched and mismatched primers, or the blocking efficiency of the two base coupling elements. The results from the experiments are shown in FIG. 3. As can be seen, when the genotype of the sample was G/G homozygous only the corresponding secondary primer, which was labeled with BFL, produced a product (FIG. 3, panel A and B, peak indicated by arrow). The secondary primer corresponding to the C allele, which was labeled with BTAMR, did not have any detectable products. When the genotype of the sample is C/C homozygous the patterns reversed, only the BTMR-labeled secondary primer produces a peak (FIG. 3, panel C and D, peak indicated by arrow). These experiments demonstrate that two consecutive mismatch bases are sufficient to block the extension of the mismatched secondary primers and do not produce unintended extension products detectable by DNA sequencers. All products observed, therefore, were directly linked to the primary reactions. The results showed that the coupling of the two AS-PCRs was highly allele-specific.

### Example 2

### Optimizing the AS-PCR² for ABI 377 DNA Sequencer Detection

ABI 377 DNA sequencers use an Argon laser as the excitation source for fluorescence detection. Because the laser has a fixed wavelength (488/516 nm) fluorophores with absorbance at longer wavelength are excited less efficiently. Although excitation can be improved by using energy transfer primers as commonly used in dye primer sequencing (24, 25), energy transfer primers were not used for this study in order to preclude a need to modify the design. Another factor that affects the signal is the filter set used in the equipment. Filters can block the signal from specified wavelength ranges. In order to obtain optimal signals for both alleles, it was necessary to optimize the ratio of the fluorescent dyes that represent the two alleles and construct dye matrices to correct spectral overlap.

Six DNA samples (of which two are homozygous for allele 1, two are heterozygous and two are homozygous for allele 2) for the SNP marker SC_{—}22 were selected, and AS-PCR² was performed with varying ratio of the two reporting dyes, BFL and BTMR. The reaction products then were analyzed by an ABI 377 DNA sequencer using GeneScan software. In the experiments the same samples were amplified and analyzed in parallel using exactly the same conditions with the exception of the ratios (BFL/BTMR) of the reporting dyes.

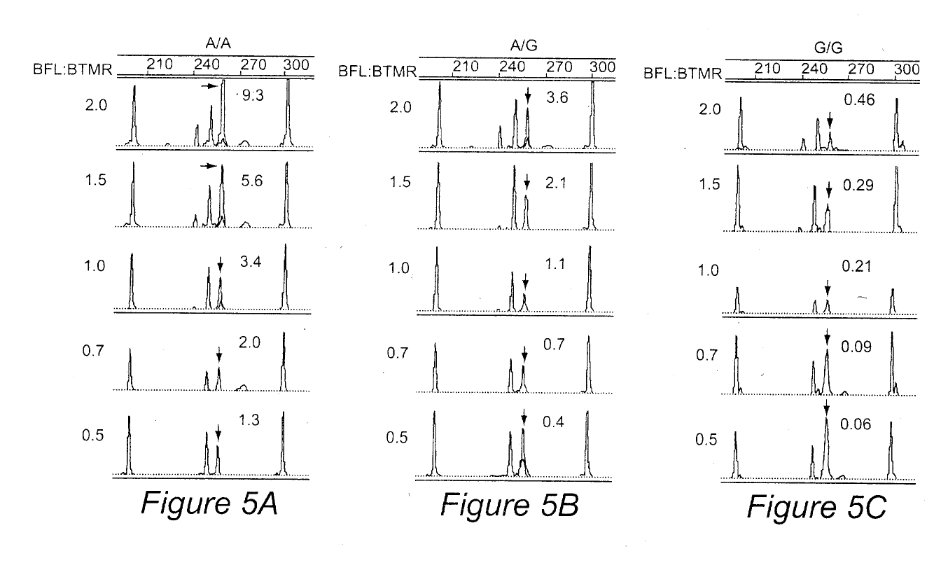

The results of the experiments were shown in FIG. 4, where samples of three known genotypes were arranged in columns and the five ratios of BFL/BTMR (2:1, 1.5:1, 1:1, 1:1.5 and 1:2, shown as 2.0, 1.5, 1.0, 0.7 and 0.5, respectively) used were arranged in rows. In the Figure, the ROX size ladder peaks are visible (GeneScan ROX500), and the BFL (dotted line) and BTMR (heavy line) peaks are indicated by arrows. The sizes are shown on the top panel of each column. For marker SC_{—}22, the expected size was 256 bases and all samples had the predicted products. For all panels, regardless the genotypes of the samples, the peak height ratio of blue/yellow peaks (as listed in each panel) decreases as the ratio of the two reporting dyes (BFL/BTMR) decreases from top to bottom. This observation suggests that the ratio of reporting dyes affected all genotypes. When the rate of change for each genotype is plotted, it was found that the rates were different for each genotype but the rate was constant for a given genotype (FIG. 5). Since the rate is a constant for a given genotype, the differences between genotype groups will also be constant. In other words genotypes could be identified correctly even if the ratios of reporting dyes are different between experiments. This becomes clear when we look at our data: the difference between the homozygous A/A (column A) and the heterozygous (column B) is about 2.5-3 fold across all panels. The difference between the heterozygous and the G/G homozygous is about 6 fold.

This experiment demonstrates that the ratio of reporting dyes affected the ratio of peak heights of the two alleles but did not change genotype scoring. One implication is that the genotype scoring was not all intuitive even when the ratio of the reporting dyes was not optimal. For example in FIG. 4, the scoring of genotype in row 3 was straightforward when the ratio of reporting dyes (1:1) was optimal. But it would be difficult to score the genotypes for rows 1 and 5 without systematic and quantitative analyses. In order to make AS-PCR² a general approach for SNP genotyping it was essential to have a sophisticated genotyping scoring algorithm.

### Example 3

### Scoring Genotypes by Secondary Primers of Different Length

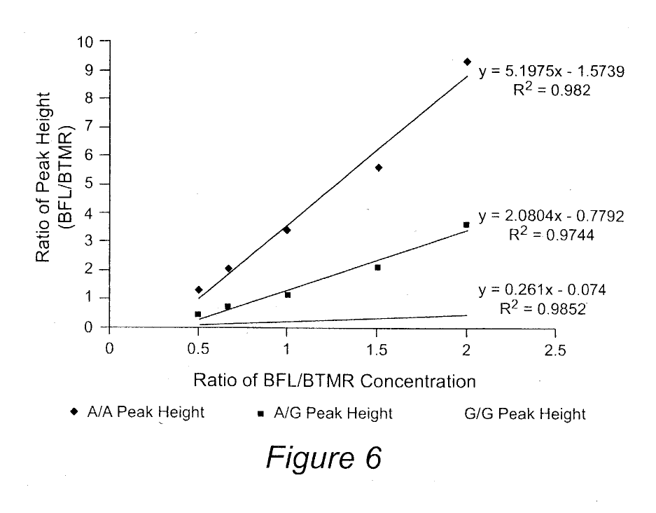

To demonstrate scoring of a genotype by the offset procedure, a secondary primer of 20 bases was synthesized and labeled with R6G (SC_{—}40). Experiments using this primer were performed with a BTMR labeled primer of 23 bases (SC 5) for the secondary reactions. Reaction products were then analyzed by GeneScan. As expected, for a heterozygote, a green peak (R6G) and a black peak (BTMR) were observed, and the peaks were offset by 3 bases, the number engineered in the allele specific secondary primers (see FIG. 6A). As comparison, a homozygote showed only one black peak.(FIG. 6B). The example shows that scoring of genotypes can be simplified in this manner.

### Example 4

### Verifying Genotyping Results by FP-TDI Assay

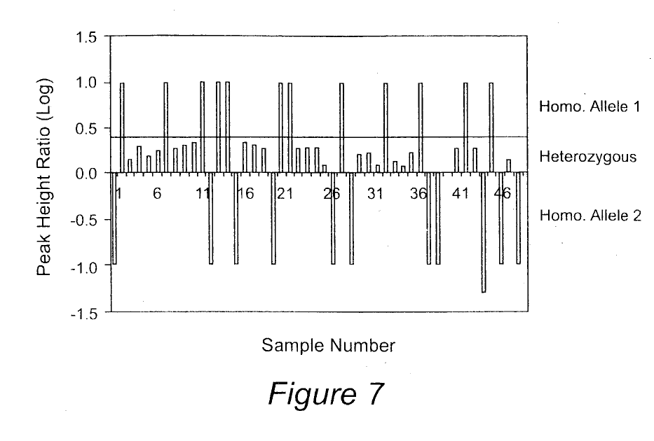

After optimization, two SNP markers, SC_{—}22 and SC_{—}31, were selected from an ongoing schizophrenia project and typed with AP-PCR² design, each for 48 subjects. FIG. 7 shows the results obtained with one of the markers, SC_{—}31. In the Figure, the logarithm value of the peak height ratio of the two alleles was plotted for each sample. The use of log values made it easier to visualize the genotypes. A large peak height ratio (>1) would be transformed into a positive log value, and a small one (<1) would be a negative. A ratio that was close to 1 would be transformed to value close to zero. When a sample had only one peak in the expected size range an arbitrary value of 10 (allele 1) or 0.1 (allele 2) was used for the peak height ratio. The plot showed three groups clearly, namely the homozygous allele 1, the heterozygous, and the homozygous allele 2. Genotypes were assigned to each sample based on which group it fell into.

The FP-TDI genotyping for the same subjects for the two markers had been previously performed about one and half years ago, and by a different individual. A comparison of the genotype results from the previous FP-TDI method with that from AS-PCR² of the present invention showed that they were in complete agreements as summarized in Table 2. For both markers, except failures in either method, all scored genotypes match each other.

TABLE 2
A comparison of genotypes between the AS-PCR2
and FP-TDI methods

| | SC_22 | | | SC_31 | | |
|---|---|---|---|---|---|---|
| Sample # | AS-PCR² | FD-TDI | Match? | AS-PCR² | FD-TDI | Match? |
| 1 | 1/2 | 1/2 | Yes | 2/2 | 2/2 | Yes |
| 2 | 1/2 | 1/2 | Yes | 1/1 | 1/1 | Yes |
| 3 | 1/2 | 1/2 | Yes | 1/2 | 1/2 | Yes |
| 4 | 0/0 | 1/2 | F | 1/2 | 1/2 | Yes |
| 5 | 1/2 | 1/2 | Yes | 2/2 | 2/2 | Yes |
| 6 | 1/2 | 1/2 | Yes | 1/1 | 1/1 | Yes |
| 7 | 1/2 | 1/2 | Yes | 0/0 | 0/0 | F |
| 8 | 1/2 | 1/2 | Yes | 0/0 | 1/2 | F |
| 9 | 1/1 | 1/1 | Yes | 1/2 | 1/2 | Yes |
| 10 | 1/1 | 1/1 | Yes | 2/2 | 2/2 | Yes |
| 11 | 1/1 | 1/1 | Yes | 1/2 | 1/2 | Yes |
| 12 | 1/2 | 1/2 | Yes | 1/2 | 1/2 | Yes |
| 13 | 1/2 | 1/2 | Yes | 1/1 | 1/1 | Yes |
| 14 | 1/2 | 1/2 | Yes | 1/2 | 1/2 | Yes |
| 15 | 1/2 | 1/2 | Yes | 1/2 | 1/2 | Yes |
| 16 | 0/0 | 1/2 | F | 1/2 | 1/2 | Yes |
| 17 | 1/2 | 1/2 | Yes | 1/1 | 1/1 | Yes |
| 18 | | 1/2 | F | 1/2 | 1/2 | Yes |
| 19 | 2/2 | 2/2 | Yes | 1/2 | 1/2 | Yes |
| 20 | 2/2 | 2/2 | Yes | 1/1 | 1/1 | Yes |
| 21 | 1/2 | 1/2 | Yes | 2/2 | 2/2 | Yes |
| 22 | 0/0 | 1/2 | F | 1/2 | 1/2 | Yes |
| 23 | 1/1 | 1/1 | Yes | 1/2 | 1/2 | Yes |
| 24 | 1/2 | 1/2 | Yes | 0/0 | 0/0 | F |
| 25 | 1/2 | 1/2 | Yes | 1/2 | 1/2 | Yes |
| 26 | 1/2 | 1/2 | Yes | 1/1 | 1/1 | Yes |
| 27 | 1/2 | 1/2 | Yes | 2/2 | 2/2 | Yes |
| 28 | 0/0 | 1/1 | F | 2/2 | 2/2 | Yes |
| 29 | 1/1 | 1/1 | Yes | 2/2 | 2/2 | Yes |
| 30 | 1/2 | 1/2 | Yes | 0/0 | 1/1 | F |
| 31 | 1/2 | 1/2 | Yes | 1/2 | 1/2 | Yes |
| 32 | 1/1 | 1/1 | Yes | 1/2 | 1/2 | Yes |
| 33 | 1/1 | 1/1 | Yes | 1/2 | 1/2 | Yes |
| 34 | 1/2 | 1/2 | Yes | 1/2 | 1/2 | Yes |
| 35 | 1/2 | 1/2 | Yes | 1/1 | 1/1 | Yes |
| 36 | 1/2 | 1/2 | Yes | 1/2 | 1/2 | Yes |
| 37 | 1/1 | 1/1 | Yes | 0/0 | 0/0 | F |
| 38 | 1/2 | 1/2 | Yes | 1/1 | 1/1 | Yes |
| 39 | 0/0 | 2/2 | F | 1/1 | 1/1 | Yes |
| 40 | 1/1 | 1/1 | Yes | 2/2 | 2/2 | Yes |
| 41 | 1/2 | 1/2 | Yes | 1/1 | 1/1 | Yes |
| 42 | 1/1 | 1/1 | Yes | 2/2 | 2/2 | Yes |
| 43 | 1/2 | 1/2 | Yes | 1/2 | 1/2 | Yes |
| 44 | 1/2 | 1/2 | Yes | 1/2 | 1/2 | Yes |
| 45 | 0/0 | 1/2 | F | 1/2 | 1/2 | Yes |
| 46 | 1/2 | 1/2 | Yes | 1/2 | 0/0 | F |
| 47 | 1/2 | 1/2 | Yes | 2/2 | 0/0 | F |
| 48 | 0/0 | 0/0 | F | 0/0 | 0/0 | F |

It was noted that the AS-PCR² method exhibited a relatively high failure rate for marker SC_{—}22 (8 vs. 1). This result was attributed to the deterioration of genomic DNA samples. After the initial genotyping of the samples, they had been stored at 4° C. and repeatedly genotyped for many markers for the schizophrenia project. When other markers were genotyped at the same time as that of AS-PCR², comparable failure rates were found. For example, for marker SC_{—}31, both methods (FP-TDI and AS-PCR²) had the same failure rate (6/48) but the samples which failed were not all the same. This finding suggests that the higher failure rate for SC_{—}22 was not likely caused by the AS-PCR² method.

These results demonstrate that AS-PCR² can be utilized in order to obtain highly accurate genotypes, the accuracy being equal to that of the traditional FP-TDI. For all 48 subjects, the genotypes obtained by the method of the present invention were in complete agreement with those obtained with the FP-TDI method.

### Example 5

### Genotype Scoring and Analysis

To score genotypes for AS-PCR, it is necessary to determine the relative quantities of the two alleles because mismatch extension does happen in the primary reactions. Peak height can be used as a measurement of the quantity of products analyzed by DNA sequencers as reported (28-30). For SNP applications, both alleles have exactly same sizes, so it is the difference of the color that provides the link to the allele specific primers. Several sets of AS-PCR² primers were designed to demonstrate the principle. A set of secondary allele specific PCR primers were designed, the two alleles were labeled with BFL (SC_{—}4) and BTMR (SC_{—}5), respectively, and AS-PCR² was performed for 48 genomic DNA samples. The reactions were run on an ABI 377 sequencer, and Genescan software was used to analyze the raw data and peak area, peak height and scan number data of each dye were exported for further analysis.

Product size was first examined. If the product size matched that which was expected, then peak color was observed, which allows the inference of genotypes. A pure blue peak (BFL) was homozygous allele 1, and a pure yellow peak (BTMR) was homozygous allele 2. When a peak had two colors, the sample could be homozygous allele 1, homozygous allele 2 or a heterozygous because color matrix correction might not be complete especially when the samples were overloaded, or a certain amount of products from mismatch extension was produced. Under these conditions robust cluster and statistic analysis would apply.

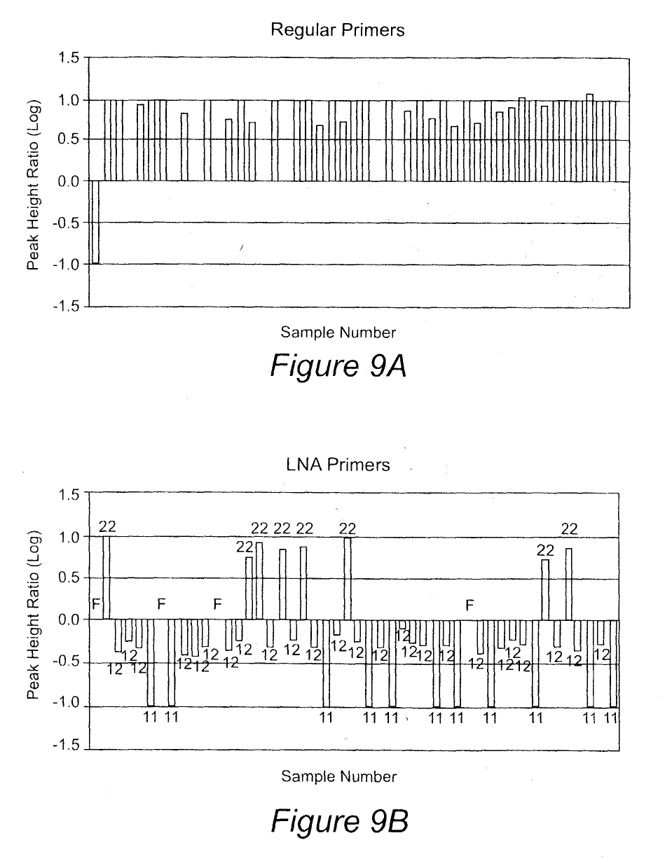

There were at least two ways to analyze the data. One was to take a ratio of intensities of the two reporting dyes (as indexed by the peak heights). Each genotype group would have a distinct ratio even if there was a small fraction of mismatch extension. (Examples are given in Example 4, FIG. 7; Example 6, FIGS. 9A and 9B; and Example 7, FIG. 11). Another way was to plot the intensities of the two reporting dyes in a two dimensional plot, and to use distance-based cluster analysis to classify the groups. To begin, an independent model was assumed, and Euclidian distances were calculated between the samples and the initial centroids of each potential group. The coordinates of the initial centers could be estimated by the frequency distribution of the samples, or assigned arbitrary. The samples were classified to a group based on their minimal distances. Then the coordinates of the centers for each group were recalculated based on the membership data points assigned from the first round classification. After several rounds of circulation the true centers of each group would be established and, used for final genotypic classification. FIG. 8 is an example of this analysis. For more sophisticated analysis, other transformed distances and covariance models can be used. For each classified sample, the posterior probability of group membership (i.e., genotype) can be calculated to provide a confidence measure for the genotypes assigned.

### Example 6

### Locked Nucleic Acid (LNA) Primers Significantly Improve Allele Discrimination for AS-PCR²

The use of LNA analog in primers could increase Tm and makes the primers hybridize to their templates more stably. The more stable LNA analogs were tested for their ability to improve the performance of AS-PCR² due to increased stability of the duplex formed with the templates. Regular and LNA modified allele specific primers using exactly the same sequences were utilized to amplify a marker known to fail AS-PCR² when regular oligonucleotide primers were used. With regular primers this marker had been tested many times under a variety of conditions and scoring of genotypes for the samples could not be accomplished. When the LNA primers were used the genotypes were clean and correct. The results were presented in FIGS. 9A and 9B, where 9A shows the results from regular primers and 9B shows the results from the LNA modified primers. As can be seen, it was not possible to score any genotypes from the reactions that used regular primers (9A). In contrast, the reactions that used the LNA primers (9B) produced 3 distinct groups, corresponding to homozygous allele 1 (labeled 11 in the figure), heterozygous (labeled 12) and homozygous allele 2 (labeled 22). The genotype results were confirmed by the FP-TDI method. These experiments demonstrate that with the use of LNA analog AS-PCR² could be very robust for SNP genotyping.

### Example 7

### Multiplex AS-PCR²

For high throughput applications multiplexing is inevitable. Two criteria are normally used to measure the success of multiplex. One is that all amplicons are amplified to generate correct products; the other is that the amounts of all amplified amplicons are relatively even. The evenness of the products is normally conditioned on the analytical tools. In the practice of the present invention, DNA sequencers may be used to score the products. Modern sequencers' detection range covers at least 3 orders of magnitude. Within this range the amount of products is correlated with the peak height. Thus, this is the window necessary to work with.

Two sets of experiments were performed to test multiplex AS-PCR². For one set, regular primers were used to multiplex 5 SNPs; for the other, LNA primers were used, also for 5 SNPs. In the regular primer set we included 2 SNPs that had been tested and worked well individually and 3 SNPs that had failed in individual marker testing. The reason to include those failed markers was to find out if multiplexing could improve them. The rationale is as follows: AS-PCR² is a kinetic process, and when it is multiplexed the competition from a different amplicon would magnify the competition between the two allele specific primers for the same amplicon. As a result, the intensified competitions should amplify the difference between the two alleles and achieve better allele discrimination.

Both sets of multiplexes were performed in the same PCR machine with exactly the same conditions. The protocol included two sequential reactions. The first reaction or primary reaction, only primary primers were used. The reactions were performed in 10 μL of volume. All primers had same concentration at 1 nM. Other components of the reactions were 500 μM of dNTPs, 2.5 mM of MgCl₂, 75 ng of genomic DNA, 0.55 units of AmpliTaq Gold DNA polymerase. After initial denaturation of 10 min at 95° C., reactions were cycled 10 times under these conditions: 95° C. for 45 sec, 65° C. for 5 sec, ramping to 55° C. at −0.1° C./sec and staying at 55° C. for 3 min. After the ten cycles, 2.5 μL of fresh enzyme-primer mix were added to each well. The mix contained 0.55 units AmpliTaq Gold DNA polymerase, 100 nM of each labeled (allele specific) secondary primer and 300 nM of reverse primer. Cycling of the reactions was resumed for 30 more times at these conditions: 95° C. for 45 sec, 55° C. for 90 sec. When the reactions were finished, 1 μL of reaction products were loaded onto a 377 DNA sequencer and analyzed by the GeneScan software.

For both sets of primers, products were obtained for all amplicons and all products had the expected sizes. Three lanes from the regular primer set are shown in FIG. 10 to illustrate two points, the evenness of products amongst the amplicons and the improvement of allele discrimination. The relative amounts of products for different amplicons varied as expected, but the difference, as measured by the peak heights, were about 10-25 fold for each lane. In the LNA set, similar variations were seen. These experiments were repeated several times and each time all products and the variation for any given lane were in a similar range. Because these multiplexing reactions were performed by standard protocol without any optimization, they prove that multiplexing AS-PCR² works, and works very well.

The peak heights listed in the figure could be used to score genotypes of the samples.

For example, for the two good marker, SC_{—}22 and SC_{—}28 (256 and 198 bp), the scoring was straight forward. For SC_{—}22, panel A and B were heterozygous (please notice that the peak height ratios were very close, 988/2789=0.35 for panel A, 364/985=0.37 for panel B), panel C was homozygous allele 1. For SC_{—}28, the peak height ratios were almost perfect: homozygous allele 1, panel C, the ratio was 0/184, for homozygous allele 2, panel B, the ration was 269/0. The heterozygous, panel A, the ratio was 1.03 (435/424).

In the multiplex experiments we observed significant improvement of allele discrimination for both LNA and regular primers. The changes for those regular primers that failed single-plex AS-PCR² were most significant. Take the example of SC_{—}25, which is the second peak from left in FIG. 10 with a product size of 216 bp. For homozygous allele 1, panel B, a ratio of 5.84 (596/102) was observed; for heterozygous, panel A, the ratio was 1.55 (1296/837), and the homozygous allele 2 had a ratio of 1.06 (428/403). Comparing these results to that shown in FIG. 9A, which were the results from single-plex AS-PCR² for the same marker, the improvement was obvious. In FIG. 9A, there were no differences between the three genotype groups. Here a clear difference between Allele 1 and the heterozygous is obvious. The peak height ratio between the heterozygous and the homozygous allele 2 was marginal, but the trend was clear as seen in FIG. 11. In the multiplexing experiments, a total of 16 samples were used. The Genotypes of the 16 samples were known, and reconfirmed by the results from LNA primers which were included for comparison. For all allele 1 homozygotes, the multiplexed regular primers gave the same results as that of the LNA primers (FIG. 11 samples had peak height ratio>0.5). The peak height ratios of heterozygotes were also correlated with that of LNA primers.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

### REFERENCES

1. Sachidanandam, R. Weissman, D., Schmidt, S. C., Kakol, J. M., Stein, L. D., Marth, G., Sherry, S., Mullikin, J. C. Mortimore, B. J., Willey, D. L., Hunt, S. E., Cole, C. G., Coggill, P. C., Rice, C. M., Ning, Z., Rogers, J., Bentley, D. R. Kwok, P. Y., Mardis, E. R. Yeh, R. T., Schultz, B., Cook, L., Davenport, R., Dante, M., Fulton, L., Hillier, L., Waterston, R. H., McPherson, J. D., Gilman, B., Schaffner, S., Van Etten, W. J., Reich, D., Higgins, J., Daly, M. J., Blumenstiel, B., Baldwin, J., Stange-Thomann, N., Zody, M. C., Linton, L., Lander, E. Atshuler, D. (2001) A map of human genome sequence variation containing 1.42 million single nucleotide polymorphisms. Nature 409, 928-933.

2. Kwok, P. Y. (2001) Methods for genotyping single nucleotide polymorphisms. Annu. Rev. Genomics Hum. Genet. 2, 235-258.

3. Kwok, P. Y. (2001) GENOMICS: Genetic Association by Whole-Genome Analysis? Science 294, 1669-1670.

4. Syvanen, A. C. (1 A.D.) Accessing genetic variation: Genotyping single nucleotide polymorphisms. Nat. Rev. Genet. 2, 930-942.

5. Bray, M. S., Boerwinkle, E., and Doris, P. A. (2001) High-throughput multiplex SNP genotyping with MALDI-TOF mass spectrometry: practice, problems and promise. Hum. Mutat. 17, 296-304.

6. Griffin, T. J. and Smith, L. M. (2000) Single-nucleotide polymorphism analysis by MALDI-TOF mass spectrometry. Trends Biotechnol. 18, 77-84.

7. Ronaghi, M. (2001) Pyrosequencing sheds light on DNA sequencing. Genome Res. 11, 3-11.

8. Livak, K. J. (1999) Allelic discrimination using fluorogenic probes and the 5′ nuclease assay. Genet. Anal. 14, 143-149.

9. Latif, S., BauerSardina, I., Ranade, K., Livak, K. J., and Kwok, P. Y. (2001) Fluorescence polarization in homogeneous nucleic acid analysis II: 5′-nuclease assay. Genome Res. 11, 436-440.

10. Mein, C. A., Barratt, B. J., Dunn, M. G., Siegmund, T., Smith, A. N., Esposito, L., Nutland, S., Stevens, H. E., Wilson, A. J., Phillips, M. S., Jarvis, N., Law, S., de Arruda, M., and Todd, J. A. (2000) Evaluation of single nucleotide polymorphism typing with invader on PCR amplicons and its automation. Genome Res. 10, 330-343.

11. Ledford, M., Friedman, K. D., Hessner, M. J., Moehlenkamp, C., Williams, T. M., and Larson, R. S. (2000) A multi-site study for detection of the factor V (Leiden) mutation from genomic DNA using a homogeneous invader microtiter plate fluorescence resonance energy transfer (FRET) assay. J. Mol. Diagn. 2, 97-104.

12. Kwok, P. Y. (2000) High-throughput genotyping assay approaches. Pharmacogenomics. 1, 95-100.

13. Myakishev, M. V., Khripin, Y., Hu, S., and Hamer, D. H. (2001) High-throughput SNP genotyping by allele-specific PCR with universal energy-transfer-labeled primers. Genome Res. 11 163-169.

14. Germer, S. and Higuchi, R. (1999) Single-tube genotyping without oligonucleotide probes. Genome Res. 9, 72-78.

15. Pastinen, T., Raitio, M., Lindroos, K., Tainola, P., Peltonen, L., and Syvanen, A. C. (2000) A system for specific, high-throughput genotyping by allele-specific primer extension on microarrays. Genome Res. 10, 1031-1042.

16. Germer, S., Holland, M. J. and Higuchi, R. (2000) High-throughput SNP allele-frequency determination in pooled DNA samples by kinetic PCR. Genome Res. 10, 258-266.

17. Ayyadevara, S., Thaden, J. J., and Shmookler Reis, R. J. (2000) Discrimination of primer 3′-nucleotide mismatch by taq DNA polymerase during polymerase chain reaction. Anal. Biochem. 284, 11-18.

18. Christensen, U., Jacobsen, N., Rajwanshi, V. K., Wengel, J., and Koch, T. (2001) Stopped-flow kinetics of locked nucleic acid (LNA)-oligonucleotide duplex formation: studies of LNA-DNA and DNA-DNA interactions. Biochem. J. 354, 481-484.

19. Kurreck, J., Wyszko, E., Gillen, C., and Erdmann, V. A. (2002) Design of antisense oligonucleotides stabilized by locked nucleic acids. Nucleic Acids Res. 30, 1911-1918.

20. Orum, H., Jakobsen, M. H., Koch, T., Vuust, J., and Borre, M. B. (1999) Detection of the factor V Leiden mutation by direct allele-specific hybridization of PCR amplicons to photoimmobilized locked nucleic acids. Clin. Chem. 45, 1898-1905

21. Hu, Y. W., Balaskas, E., Kessler, G., Issid, C., Scully, L. J., Murphy, D. G., Rinfret, A., Giulivi, A., Scalia, V., and Gill, P. (1998) Primer specific and mispair extension analysis (PSMEA) as a simple approach to fast genotyping. Nucleic Acids Res. 26, 5013-5015.

22. RehaKrantz, L. J., Stocki, S., Nonay, R. L., Dimayuga, E., Goodrich, L. D., Konigsberg, W. H., and Spicer, E. K. (1991) DNA polymerization in the absence of exonucleolytic proofreading: in vivo and in vitro studies. Proc. Natl. Acad. Sci. U.S. A 88, 2417-2421.

23. Chen, X., Levine, L., and Kwok, P. Y. (1999) Fluorescence polarization in homogeneous nucleic acid analysis. Genome Res. 9, 492-498.

24. Ju, J., Glazer, A. N., and Mathies, R. A. (1996) Cassette labeling for facile construction of energy transfer fluorescent primers. Nucleic Acids Res. 24, 1144-1148.

25. Ju, J., Glazer, A. N., and Mathies, R. A. (1996) Energy transfer primers: a new fluorescence labeling paradigm for DNA sequencing and analysis. Nat. Med. 2, 246-249.

26. Nielsen K E, Singh S K, Wengel J, Jacobsen J P. 2000. Solution structure of an LNA hybridized to DNA: NMR study of the d(CT(L)GCT(L)T(L)CT(L)GC):d(GCAGAAGCAG) duplex containing four locked nucleotides. Bioconjug. Chem. 11:228-38

27. Petersen M, Nielsen C B, Nielsen K E, Jensen G A, Bondensgaard K, Singh S K, Rajwanshi V K, Koshkin A A, Dahl B M, Wengel J, Jacobsen J P. 2000. The conformations of locked nucleic acids (LNA). J. Mol. Recognit. 13:44-53

28. Hoogendoorn B, Norton N, Kirov G, Williams N, Hamshere M L, Spurlock G, Austin J, Stephens M K, Buckland P R, Owen M J, O'Donovan M C. 2000. Cheap, accurate and rapid allele frequency estimation of single nucleotide polymorphisms by primer extension and DHPLC in DNA pools. Hum. Genet. 107:488-93

29. Daniels J, Holmans P, Williams N, Turic D, McGuffin P, Plomin R, Owen M J. 1998. A simple method for analyzing microsatellite allele image patterns generated from DNA pools and its application to allelic association studies. Am. J. Hum. Genet. 62:1189-97

30. Barcellos L F, Klitz W, Field L L, Tobias R, Bowcock A M, Wilson R, Nelson M P, Nagatomi J, Thomson G. 1997. Association mapping of disease loci, by use of a pooled DNA genomic screen. Am. J. Hum. Genet. 61:734-47

31. Braasch D A, Corey D R 2001. Locked nucleic acid (LNA): fine-tuning the recognition of DNA and RNA. Chem. Biol. 8:1-7

32. Delahunty C, Ankener W, Deng Q, Eng J, Nickerson D A. 1996. Testing the feasibility of DNA typing for human identification by PCR and an oligonucleotide ligation assay. Am. J. Hum. Genet. 58:1239-46

We claim:

1. A method of genotyping one or more loci in a DNA sample, comprising the steps of:
combining
a sample containing single stranded DNA or double stranded DNA,
at least one primary primer specific for one locus on one strand of DNA in said sample, said primary primer having a first homologous portion which hybridizes to said one strand of DNA and a non-homologous portion which does not hybridize to said one strand of DNA,
at least one secondary primer having a second homologous portion which comprises sequences identical to those of said non-homologous portion of said primary primer;
conducting polymerase chain reaction (PCR); and
identifying amplicons of said PCR which include said non-homologous portion, wherein said step of identifying allows the genotype of said one or more loci to be established.

2. The method of claim 1 wherein said combining step is performed using a plurality of primary primers, wherein each of said plurality of primary primers is specific for a different locus in said DNA sample, and includes
a homologous portion which is different for each primary primer in said plurality of primary primers so that each primary primer hybridizes to said DNA at a different locus, and
an identical non-homologous portion.

3. The method of claim 1 wherein said amplicons are identified by a technique selected from the group consisting of electrophoresis, microfluidics, microarray or chip detection, fluorescence polarization, fluorescence resonance energy transfer, and mass spectrometry.

4. The method of claim 1 wherein said locus contains a detectable distinguishing feature selected from the group consisting of an SNP, a deletion, an insertion, and a short tandem repeat.

5. The method of claim 1 wherein said secondary primer further comprises a detectable label selected from the group consisting of fluorescent dyes, antibodies, enzymes, magnetic moieties, electronic markers, and mass tags.

6. The method of claim 1 wherein said at least one primary primer and said at least one secondary primer comprise locked nucleic acids.

7. The method of claim 1 wherein said secondary primers have a different length for each allele.

8. A primer set for genotyping one or more loci in a DNA sample, comprising,
at least one primary primer specific for one locus on one strand of DNA in said sample, said primary primer having a first homologous portion which hybridizes to said one strand of DNA and a non-homologous portion which does not hybridize to said one strand of DNA, and
at least one secondary primer having a second homologous portion which comprises sequences identical to those of said non-homologous portion of said primary primer.

9. The primer set of claim 8 wherein said secondary primer further comprises a detectable label selected from the group consisting of fluorescent dyes, antibodies, enzymes, magnetic moieties, electronic markers, and mass tags.

10. A method of multiplex PCR for a plurality of loci in a DNA sample, comprising the steps of:
carrying out a first round of PCR amplification with at least one primary primer specific for one locus on one strand of DNA in said sample, said primary primer having a first homologous portion which hybridizes to said one strand of DNA and a non-homologous portion which does not hybridize to said one strand of DNA, wherein said primary primers are present in an equal and limited amount, and
carrying out a second round of amplification with at least one secondary primer having a second homologous portion which comprises sequences identical to those of said non-homologous portion of said primary primer; wherein said secondary primers are present in a non-limiting amount.

11. The method of claim 10 wherein said first round of PCR employs limited cycling.

12. A kit, comprising
instructions for the design of primary primers having a first homologous portion which hybridizes to one strand of DNA and a non-homologous portion which does not hybridize to said strand of DNA, and
secondary PCR primers having a second homologous portion which comprises sequences identical to those of said non-homologous portion of said primary primer.
